(19) United States
(12) Patent Application Publication
(10) Pub. No.: US 2025/0290935 A1
(43) Pub. Date: Sep. 18, 2025

(54) MARKER OF ALZHEIMER'S DISEASE AND USE THEREOF

(71) Applicant: SHENZHEN INSTITUTES OF ADVANCED TECHNOLGY CHINESE ACADEMY OF SCIENCES, Shenzhen (CN)

(72) Inventors: Yu CHEN, Shenzhen (CN); Xueqi QU, Shenzhen (CN)

(73) Assignee: SHENZHEN INSTITUTES OF ADVANCED TECHNOLGY CHINESE ACADEMY OF SCIENCES, Shenzhen (CN)

(21) Appl. No.: 18/575,186

(22) PCT Filed: Jun. 30, 2021
(86) PCT No.: PCT/CN2021/103482

§ 371 Date: Jan. 18, 2024

(51) Int. Cl. G01N 33/68

(52) U.S. Cl. CPC G01N 33/6896; G01N 33/6863; G01N 2333/523; G01N 2800/2821

(57) ABSTRACT

Provided in the present application is a marker of Alzheimer's disease, which marker comprises monocyte chemoattractant protein-1 (MCP 1). Further provided in the present application are a method for detecting the marker of Alzheimer's disease, a kit for detecting Alzheimer's disease, and the use of the marker, the detection method and the kit in the screening of a drug for treating Alzheimer's disease.

### CROSS-REFERENCE TO RELATED APPLICATION

This is a National stage application, filed under 37 U.S.C. 371, of International Patent Application No. PCT/CN2021/103482, filed on Jun. 30, 2021.

### INCORPORATION-BY-REFERENCE OF MATERIAL SUBMITTED ON A READ-ONLY OPTICAL DISC, AS A TEXT FILE OR AN XML FILE VIA THE PATENT ELECTRONIC SYSTEM

The ASCII plain text file entitled 963P180sequence.txt dated Jun. 7, 2024 and having a size of 628 bytes containing a Sequence Listing is hereby incorporated by reference.

### TECHNICAL FIELD

The present application belongs to the field of molecular detection and in particular, relates to a marker of Alzheimer's disease and use thereof.

### BACKGROUND

Alzheimer's disease (AD) is an age-related neurodegenerative disease in the central nervous system and is the most common dementia among the elderly. Clinically, patients with AD have features such as memory loss, cognitive impairment, language impairment, decreased executive ability, and personality changes. Typical pathological features of patients with AD are oligomeric beta amyloid protein (AB) plaque aggregation, neurofibrillary tangles (NFTs) formed by the hyperphosphorylation of Tau proteins, and various inflammatory responses in the brain.

The etiology of AD is complex, and the pathogenesis of AD is still unknown. Currently, the widely accepted hypotheses of the cause of the disease include the AB aggregation hypothesis, the Tau protein abnormality hypothesis, the cholinergic hypothesis, the neuroinflammation hypothesis, and the like. It is to be noted that no matter what the hypothesis is, the brains of patients with AD are accompanied by inflammatory responses, and studies have shown that neuroinflammation occurs before the formation of AB plaques and NFTs. Because of the complex etiology of AD and the unclear pathogenesis of AD, the progress of drug research is slow, and there is still a lack of drugs or methods to cure AD worldwide.

The course of AD is an irreversible process; therefore, the key to the treatment of AD is early diagnosis to intervene in AD in the early stages and delay its progression. However, there has been no precise method for early prediction or diagnosis of AD so far. Currently, the diagnosis method of AD is mainly combined diagnosis and mainly includes: neuropsychological assessment, cognitive impairment testing, positron emission tomography (PET) scanning of the brain of the elderly for plaques and Tau proteins, magnetic resonance imaging (MRI) of the brain and detection of markers in cerebrospinal fluid (CSF), Aβ detection, and phosphorylated Tau protein detection. However, these techniques are either expensive or at risk of intrusive infection, and it is not practical to apply these methods to the disease routine screening.

Therefore, how to provide an early screening method with easy operation, lower cost and higher safety for AD has become an urgent problem to be solved.

### SUMMARY

The present application provides a marker of Alzheimer's disease and use thereof, in which the level of inflammation in the brain is evaluated by detecting the expression level of a chemokine monocyte chemoattractant protein 1 (MCP1) in the peripheral blood to further assist in an early screening of AD.

In a first aspect, the present application provides a marker of Alzheimer's disease, and the marker of Alzheimer's disease includes a chemokine MCP1.

In the present application, MCP1 is monocyte chemotactic protein 1, has chemotactic activity on monocytes, can activate monocytes and macrophages to release cytokines, and thus can be used to evaluate the inflammatory level of a body. Studies have shown that the inflammation level in patients with AD significantly increases, indicating that the peripheral immune system may play an important role in the pathogenesis of AD. Therefore, the expression level of MCP1 may be used as a screening marker for AD. When the expression level of MCP1 is used as a marker, peripheral blood may be directly detected without the cooperation with professional instruments, which is easy to operate, less expensive and safer and thus is of practical significance.

In a second aspect, the present application provides a method for detecting Alzheimer's disease for the purpose of non-disease diagnosis and/or treatment, and the method includes the step of detecting MCP1 of a sample.

In the present application, the detection of MCP1 in a sample may be used in the early screening of AD, which is convenient in sampling and simple to operate, has less damage to the sample and lower risk, and thus has clinical significance.

Preferably, the sample includes cerebrospinal fluid and/or serum.

Preferably, the step of detecting MCP1 of the sample includes: detecting an expression level of MCP1 and/or determining a location of MCP1.

Preferably, a method for detecting the expression level of MCP1 includes real-time fluorescence quantitative polymerase chain reaction (PCR) detection and/or enzyme-linked immunosorbent assay (ELISA) detection.

Preferably, a method for determining the location of MCP1 includes immunofluorescence staining.

Preferably, the method for detecting Alzheimer's disease for the purpose of non-disease diagnosis and/or treatment further includes a step of detecting any one or a combination of at least two of an activity of regulatory T cells, an activity of T helper cells, an aggregation level of Aβ plaques, an expression level of inflammatory factors or an expression level of cytokines of the sample.

In a third aspect, the present application provides a kit for detecting Alzheimer's disease. The kit detects the marker of Alzheimer's disease described in the first aspect. The kit includes an agent for detecting the marker of Alzheimer's disease described in the first aspect.

The kit includes any one of an immunofluorescence staining kit, a real-time fluorescence quantitative PCR kit or an ELISA kit.

In the present application, with the use of a corresponding kit, the detection result is more accurate and the operation is simpler, thereby facilitating the promotion of related technology and methods.

Preferably, the immunofluorescence staining kit includes any one or a combination of at least two of a fixing agent, a dehydrating agent, an embedding agent, a cleaning liquid, a sealing agent, a primary antibody, a secondary antibody or a mounting medium.

Preferably, the immunofluorescence staining kit includes a fixing agent, a dehydrating agent, an embedding agent, a cleaning liquid, a sealing agent, a primary antibody, a secondary antibody, and a mounting medium.

Preferably, the real-time fluorescence quantitative PCR kit includes any one or a combination of at least two of an RNA extraction reagent, a reverse transcriptase, an amplification enzyme, a buffer, a primer, an RNA enzyme inhibitor, dNTPs, or a fluorescent dye.

Preferably, the real-time fluorescence quantitative PCR kit includes an RNA extraction reagent, a reverse transcriptase, an amplification enzyme, a buffer, a primer, an RNA enzyme inhibitor, dNTPs, and a fluorescent dye.

Preferably, the ELISA kit includes any one or a combination of at least two of an MCP1 conjugate, a luminescent reagent, a positive control, a standard substance, a diluent, a scrubbing solution or a stop solution.

Preferably, the ELISA kit includes an MCP1 conjugate, a luminescent reagent, a positive control, a standard substance, a diluent, a scrubbing solution, and a stop solution.

In a fourth aspect, the present application provides the use of any one or a combination of at least two of the marker of AD described in the first aspect, the method for detecting AD for the purpose of non-disease diagnosis and/or treatment described in the second aspect or the kit for detecting AD described in the third aspect in the screening of a drug for treating AD.

Compared with the prior art, the present application has the following beneficial effects.

In the present application, a chemokine MCP1 is used as a marker of AD so that the peripheral blood sample can be directly detected to determine the inflammation level of the body, which is used for the early screening of AD with a simple operation and low risk. With the use of a corresponding kit, the detection efficiency is improved, the accuracy of the result is increased, and the detection is of value in practical application, can be used to guide clinical screening, the research of AD pathogenesis, and the screening of a drug for treating AD, and has a wide application prospect.

### BRIEF DESCRIPTION OF DRAWINGS

FIG. 1 is a picture of results of immunofluorescence staining of brain tissues of 6-month-old, 9-month-old, and 12-month-old wild-type mice and APP/PS1 mice according to Example 1 of the present application (scale=50 μm);

FIG. 2A is a statistical graph of percentages of different cells in the gate of PBMCs of 3-month-old wild-type mice and APP/PS1 mice detected by flow cytometry according to Example 2 of the present application;

FIG. 2B is a statistical graph of percentages of different cells in the gate of PBMCs of 6-month-old wild-type mice and APP/PS1 mice detected by flow cytometry according to Example 2 of the present application;

FIG. 3 is a picture of results of immunofluorescence staining of brain tissues of 6-month-old wild-type mice and APP/PS1 mice according to Example 3 of the present application (scale=50 μm);

FIG. 4A is a picture of results of the expression level of MCP1 in the cortex and hippocampus of 3-month-old wild-type mice and APP/PS1 mice according to Example 4 of the present application;

FIG. 4B is a picture of results of the expression level of MCP1 in the cortex and hippocampus of 6-month-old wild-type mice and APP/PS1 mice according to Example 4 of the present application;

FIG. 5A is a picture of results of changes in expression amounts of MCP1 in the cerebrospinal fluid of 3-month-old and 6-month-old wild-type mice and APP/PS1 mice according to Example 5 of the present application; and

FIG. 5B is a picture of results of changes in expression amounts of MCP1 in the peripheral blood of 3-month-old and 6-month-old wild-type mice and APP/PS1 mice according to Example 5 of the present application.

### DETAILED DESCRIPTION

To further elaborate on the technical means adopted and effects achieved in the present application, the present application is further described below in conjunction with examples and drawings. It is to be understood that the specific examples set forth below are intended to explain the present application and are not to limit the present application.

Experiments without specific techniques or conditions specified in the examples are conducted according to techniques or conditions described in the literature in the art or according to product specifications. The reagents or instruments used herein without manufacturers specified are conventional products commercially available from proper channels.

### Materials:

wild-type and APP/PS1 mouse models, obtained from the Jackson Laboratory, the United States;
paraformaldehyde, purchased from Sigma-Aldrich, Cat. No.: 158127;
an embedding agent OCT, purchased from SAKURA, Cat. No.: 4583;
Iba1 primary antibody, purchased from Wake Company, Cat. No.: 019-19741;
CD8 primary antibody, purchased from Invitrogen, Cat. No.: 14-0195-82;
Aβ primary antibody, purchased from Biolegend, Cat. No.: 800717;
MCP1 primary antibody, purchased from Abcam, Cat. No.: ab7202;
fluorescence secondary antibody, purchased from Thermo Scientific;
red blood cell lysis buffer, purchased from BD Biosciences, Cat. No.: 555899;
antibodies used in flow cytometry, purchased from BD Biosciences:
Ms CD45 FITC 30-F11, Cat. No.: 553079;
Ms CD3 MolCpx PerCP-Cy5.5 17A2, Cat. No.: 560527;
Ms CD4 APC-H7 GK1.5, Cat. No.: 560181;
Ms CD8a PE 53-6.7, Cat. No.: 553032;
Ms CD19 PE-Cy7 1D3, Cat. No.: 552854;
Ms CD49b APC DX5, Cat. No.: 560628;
horse serum, purchased from Gibco, Cat. No.: 26050088;
fetal bovine serum, purchased from Life Technologies, Cat. No.: 16050-122;
DAPI, purchased from Thermo Scientific, Cat. No.: D1306;
DPBS, purchased from Sigma, Cat. No.: D8662-24*500ML;
Trizol, purchased from Invitrogen, Cat. No.: 15596026;
reverse transcription kit, purchased from Thermo Scientific, Cat. No.: K1622;
real-time fluorescence quantitative PCR kit, purchased from Thermo Scientific, Cat. No.: 4368706;
ELISA kit, purchased from R&D, Cat. No.: MJE00B.

### Example 1

In this example, the immunofluorescence staining kit was used for MCP1 and AB staining of brain tissues of wild-type mouse models and APP/PS1 mouse models (AD mouse models), and the steps are as follows:

### 1. Mouse Brain Tissue Slice

(1) Anesthesia and brain tissue perfusion sampling: The mice each were anesthetized by intraperitoneal injection of chloral hydrate. After deep anesthesia, the mice each were fixed on an operating board and placed in a dissecting tray. The brain tissue was collected from the back of the head and then soaked and fixed in paraformaldehyde for 24 hours.

(2) Perfusion fixation of mice: The mice each were perfused with PBS at 4° C., 20 mL per mouse, and then perfused with 4% paraformaldehyde at 4° C. (40 g of paraformaldehyde was added to a glass container containing 500 mL of DEPC water, heated continuously, and stirred magnetically until the temperature reached 60° C. to form a milky white suspension, the pH of the suspension was adjusted to 7.0 using 1.0 mmol/L NaOH to make the solution clear, and about 500 mL of 2×PBS was added to the solution, mixed thoroughly, filtered, filled to 1000 mL, and stored at 4° C. for later use), 20 mL per mouse, until the tissue was hardened.

(3) Tissue collection: The brain tissue was carefully peeled off, placed into a 15 mL centrifuge tube, and fixed using 4% paraformaldehyde (fixing agent) for 24 hours.

(4) Dehydration: The fixed tissue was washed three times with PBS (cleaning solution), dehydrated using 20% sucrose (dehydrating agent) until the tissue sank down completely, and then dehydrated using 30% sucrose at 4° C. overnight.

(5) The OCT embedding agent was added onto a specimen holder dropwise and placed inside a cryostat microtome until the specimen turned white. The specimen holder was taken out, and the surface of the specimen was quickly leveled using a single-edged blade.

(6) After the bottom of the specimen was leveled using a safety-razor blade, the specimen was attached to a specimen holder and placed into a freezer of the cryostat microtome at −24° C. When the tissue was slightly whitened, a thin layer of OCT was applied onto the surface of the specimen and then frozen for 20 minutes.

(7) After the slice thickness was adjusted, the tissue was sliced, with a slice thickness of 20 μm, and the slices were continuously collected and transferred into a 24-well plate containing 4% paraformaldehyde.

(8) The obtained slices were stored at 4° C. for later use.

### 2. Immunofluorescence Staining

(1) Slices at appropriate positions were picked out, placed into a 24-well plate containing 1 mL of pre-cooled PBS, and washed using the pre-cooled PBS three times for 10 minutes each time.

(2) Punching and sealing: The slices were incubated using 0.2% Triton X-100 (diluted with PBS), 0.1% BSA, and 5% horse serum (diluted with PBS) at room temperature for 40 minutes, and shaken slowly on a shaker.

(3) The slides were washed using PBS at room temperature three times for 5 minutes each time.

(4) Primary antibody incubation: The slices were diluted using an antibody diluent (PBS containing 0.01% BSA and 5% horse serum) at 1:100, 200 μL per well, and incubated while slowly shaking at 4° C. overnight.

(5) Primary antibody was recovered, and the slices were washed using PBS at room temperature three times for 10 minutes each time.

(6) The slices were sealed using 3% horse serum at room temperature for 30 minutes.

(7) Second antibody incubation and DAPI staining: The slices were diluted using PBS at 1:500 and then incubated at room temperature for 2 hours, the storage solution was diluted using DAPI at 1:5000, and then incubated at room temperature for 15 minutes.

(8) The slices were washed using PBS at room temperature three times for 5 minutes each time.

(9) Sealing: A sticky slide was marked with the specific information with a pencil on the grinding surface on the right side, a drop of PBS was dropped in the middle of the slide, a slice was dipped and placed on the drop of PBS, the PBS solution was aspirated, 160 μL of mounting medium was spread across the center of the slide, the slice was covered using a long coverslip to avoid bubbles and wrinkles.

(10) The slide was dried flat away from light.

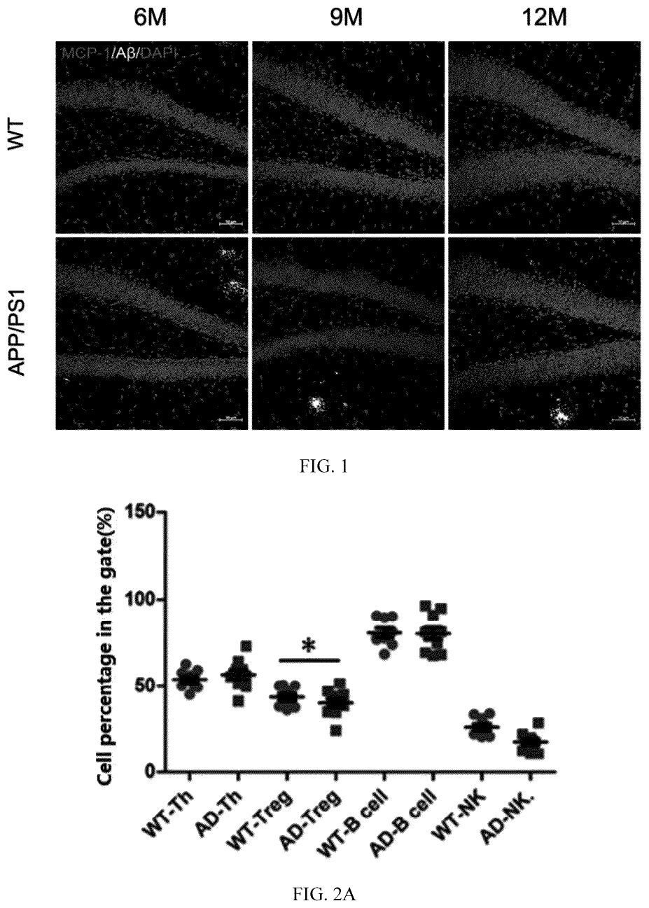

The staining pictures of 6-month-old (6 M), 9-month-old (9 M), and 12-month-old (12 M) wild-type mice and APP/PS1 mice are shown in FIG. 1. As can be seen from the figure, MCP1 occurred in the cortex and hippocampus of 6-month-old AD mice; with the progression of the disease, the expression of MCP1 was increased in the brain tissues of 9-month-old and 12-month-old AD mice, and Aβ plaques were increased in number and size and became aggregated; while no MCP1 occurred in the 3-month-old mice. Such a result indicates that in AD mouse models, MCP1-mediated neuroinflammation occurred between 3 and 6 months of age or earlier, which indicates that MCP1 can be used as an early marker of AD.

### Example 2

In this example, flow cytometry was performed on the peripheral blood mononuclear cells (PBMCs) of 6-month-old and 9-month-old wild-type mice and APP/PS1 mice, and the steps are as follows:

### 1. Preparation of Highly Active Peripheral Blood Mononuclear Cells (PBMCs)

(1) Three volumes of red blood cell lysis buffer were added to 200 μL of anticoagulant whole blood, mixed gently, and allowed to stand at room temperature for 10 minutes during which the mixture was mixed gently twice, to lyse red blood cells.

(2) The mixture was centrifuged at 800×g for 2 minutes, the supernatant was discarded, the cells were collected, and the sample was washed using 1 mL of PBS once.

(3) The cells were resuspended using 500 μL of buffer and filtered using a 300-mesh cell strainer, and an antibody was incubated and then analyzed on a flow cytometer.

### 2. Flow Cytometry

(1) The PBMC cell suspension prepared in the above step was adjusted to a density of 5× 106 cells/mL using DPBS containing 2% fetal bovine serum.

(2) 40 μL of cell suspension was added to a plastic centrifuge tube to which 50 μL of fluorescence-labeled specific antibody was pre-added, 50 μL of inactivated normal horse serum (dilute with DPBS at 1:20) was added, and the cells were incubated at 4° C. for 30 minutes.

(3) The cells were mixed thoroughly and washed by adding 2 mL of DPBS containing 2% fetal bovine serum, centrifuged at 1000 rpm for 5 minutes at 4° C., and washed once again.

(4) The cells were resuspended by adding 500 μL of pre-cooled PBS to prepare for flow cytometry.

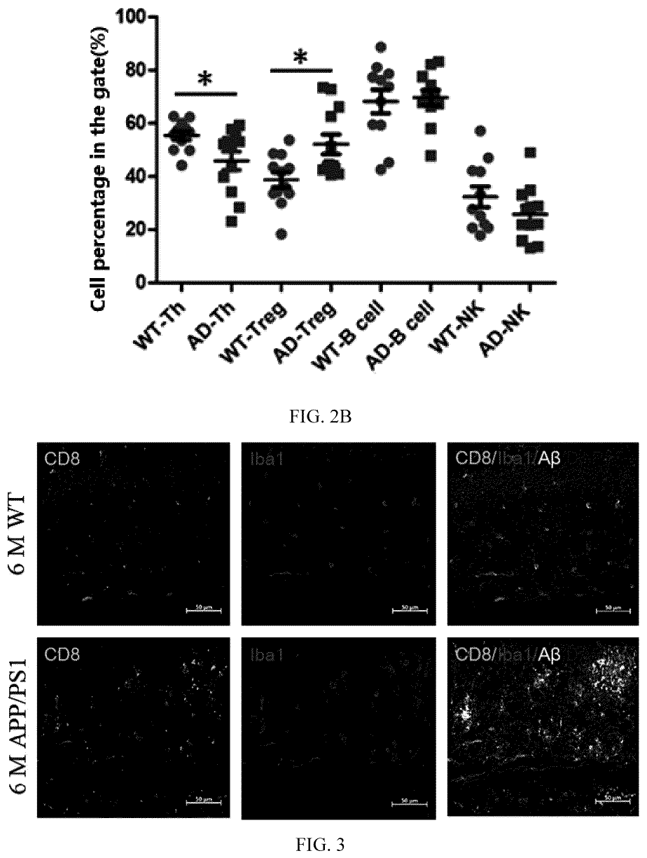

The percentages of regulatory T cells (Treg), T helper cells (Th), B cells, and natural killer cells (NK) in different cell gates were counted, respectively, and the statistical results are shown in FIGS. 2A and 2B.

As can be seen from the figures, the activity of Treg cells was significantly inhibited in 3-month-old mice, and as the disease progressed, the activity of Treg cells was increased significantly in the peripheral blood of 6-month-old mice. It is suggested that in the early stage of AD, the body's immune response was activated, the activity of Treg cells was inhibited, and the immunosuppressive effect of Treg cells on the body was reduced, indicating that Treg cells in the peripheral blood may play an inhibitory role in neuroinflammation in the brain in the early stage of AD. As AD progressed, the activity of Treg cells was significantly activated, the immunosuppressive effect of Treg cells on the body was enhanced, and the immune response of the body was reduced, which may exacerbate neuroinflammation in the brain. In addition, the activity of Th cells was significantly decreased in 6-month-old mice as AD progressed.

### Example 3

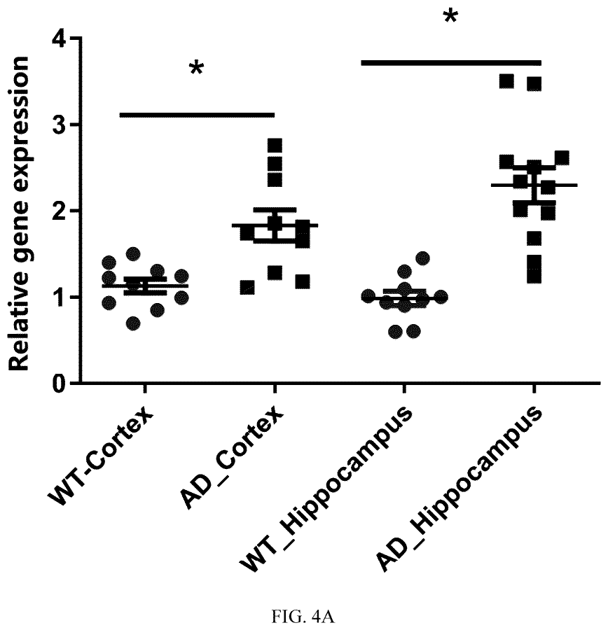

In this example, immunofluorescence staining was performed on brain tissues of 6-month-old wild-type mice and APP/PS1 mice, and CD8+ T cell markers Iba1 and CD8 are co-stained with Aβ. Experimental steps are the same as those in Example 1, and the results are shown in FIG. 3.

As can be seen from the figure, CD8+ T cells were aggregated around the Aβ plaques, which indicates that CD8+ T cells showed chemotaxis in sites of neuroinflammation in the brain and around the AB plaques, revealing that CD8+ T cells have a regulatory role in Aβ plaque-mediated neuroinflammation in the brain.

### Example 4

In this example, the expression level of MCP1 in the cortex and hippocampus of 3-month-old and 6-month-old wild-type mice and APP/PS1 mice was detected using real-time fluorescence quantitative PCR kits, respectively, and the steps are as follows.

### 1. RNA Extraction

(1) 3-month-old and 6-month-old wild-type mice and their AD little mates were anesthetized with isoflurane (gas), sacrificed by breaking their necks, and rapidly decapitated with scissors, the heads were on ice, the cerebral cortex was rapidly separated, and the cortex and hippocampus of the mice were separated. The separated tissues each were washed twice with DPBS containing 4 U/mL protease inhibitor and RNA enzyme inhibitor.

(2) Homogenization processing: The tissue or cells was ground in liquid nitrogen, 1 mL of Trizol (RNA extraction reagent) was added for every 100 mg of tissue, and homogenization processing was performed using a homogenizer.

(3) The homogenized sample was allowed to stand at room temperature for 5 minutes to completely separate the nucleic acid protein complex.

(4) 0.2 mL chloroform was added for every 1 mL of Trizol, and the sample was severely oscillated for 15 seconds and allowed to stand at room temperature for 3 minutes.

(5) The sample was centrifuged at 10000×g for 15 minutes at 4° C.

(6) The aqueous phase was transferred to a new tube, the RNA in the aqueous phase was precipitated using isopropanol, 0.5 mL of isopropanol was added for every 1 mL of Trizol, and allowed to stand at room temperature for 10 minutes.

(7) The aqueous phase was centrifuged at 10000×g for 10 minutes at 4° C. until a colloid precipitate occurred on the wall and bottom of the tube, and the supernatant was discarded.

(8) The RNA precipitate was washed using 75% ethanol, 1 mL of 75% ethanol was added for every 1 mL of Trizol, the RNA precipitate was centrifuged at 7500×g for 5 minutes at 4° C., and the supernatant was discarded.

(9) The RNA precipitate was dried at room temperature for 5 minutes, 50 μL of RNase-free water was added, the RNA precipitate was pipetted with a tip several times, allowed to stand at 55° C. for 10 minutes to dissolve the RNA, and stored at −70° C.

### 2. Reverse Transcription

The extracted total RNA was reverse transcribed into cDNA using a reverse transcription kit, and the steps are as follows.

The reaction mixture I was prepared in an RNA enzyme-free centrifuge tube using the following system:
Component Amount
Total RNA 1μ g
Oligo (dT) 1 ML
Random primer 1 ML
RNA enzyme-free water add to 12 μL
Total volume 12 μL

The above components were mixed, rapidly centrifuged for 5 seconds, incubated at 70° C. for 5 minutes, and placed in an ice bath for 2 minutes. The reaction mixture II was prepared using the following system:
Component Volume (μL)
Buffer 4
RNA enzyme inhibitor 1
dNTPs (10 mM) 2
Reverse transcriptase 1
Total volume 8

The reaction mixture I was added to the reaction mixture II, mixed rapidly for 5 seconds, incubated at 70° C. for 5 minutes, and placed in an ice bath for 2 minutes. Reverse transcription was performed according to the following procedure:
25° C. for 5 minutes; 42° C. for 60 minutes; 70° C. for 5 minutes.

The resulting cDNA template was stored at −20° C. for later use.

### 3. Real-Time Fluorescence Quantitative PCR

The expression level of MCP1 was detected using real-time fluorescence quantitative PCR kits, and the reaction system is as follows:
Component Volume (μL)
Forward primer (10 mM) 1
Reverse primer (10 mM) 1
Mixture of enzyme, buffer, dNTPs, and dye 10
cDNA template 1
Deionized water 7
Total volume 20

The sequence of the forward primer was shown in SEQ ID No. 1, and the sequence of the reverse primer was shown in SEQ ID No. 2:
SEQ ID No. 1: aggtgtcccaaagaagctgt;
SEQ ID No.2: acagaagtgcttgaggtggt.

The above components were mixed, centrifuged at 6000 rpm for 1 minute, and then amplified according to the following procedure:
pre-denaturation: 95° C. for 10 minutes;
cyclic amplification: 95° C. for 15 seconds; 60° C. for 1 minute; 70° C. for 1 minute;
for 40 cycles;
melting curve formation: 95° C. for 15 seconds; 60° C. for 1 minute.

During the whole process, the heating and cooling rates were 1.6° C./s.

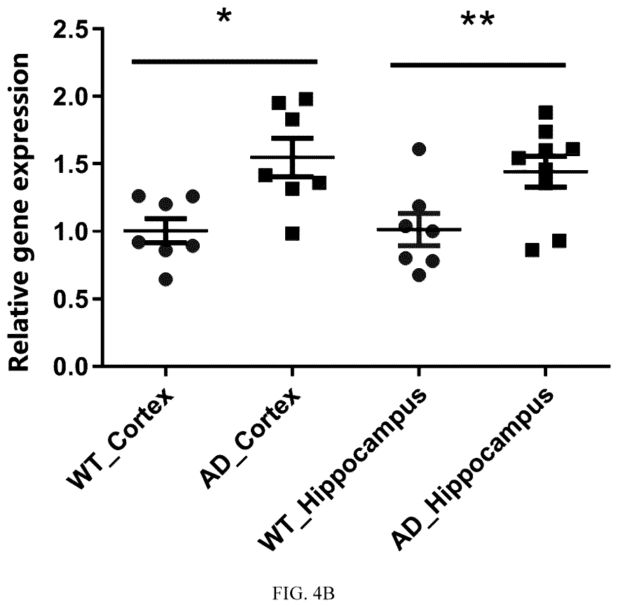

The detection results are shown in FIGS. 4A and 4B.

As can be seen from the figures, compared with 3-month-old and 6-month-old AD mice, the changes in cytokine expression between individuals in the same group of wild-type mice were relatively concentrated; while the changes in cytokine expression between different individuals in the same group of AD mice varied considerably, and the gene expression level of MCP1 was significantly increased in the cortex and hippocampus of 3-month-old and 6-month-old AD model mice.

### Example 5

In this example, the expression level of MCP1 in the cerebrospinal fluid and peripheral blood of 3-month-old and 6-month-old wild-type mice and APP/PS1 mice was detected using ELISA kits, respectively, and the steps are as follows.

### 1. Sample Collection

### (1) Serum Sample Collection

3-month-old and 6-month-old wild-type mice and their AD little mates were anesthetized with isoflurane gas, the blood sample of each mouse was collected into a 1.5 mL sterilized EP tube from the fundus, the mice each were sacrificed by breaking their necks and rapidly decapitated with scissors, and the serum was separated according to the following method.

The anticoagulant sample was allowed to stand at 4° C. for 4 hours, the serum was naturally precipitated after the blood was clotted, centrifuged at 4000 rpm for 30 minutes at 4° C., and separated, and the insoluble material was discarded.

The serum was transferred to a new sterilized EP tube and stored at −80° C.

### (2) Cerebrospinal Fluid Sample Collection

The mice each were anesthetized, their heads were fixed on the stereotaxic apparatus. When the cerebrospinal fluid was collected, the skin of the back and neck was wiped with a wet gauze, the back hair was cut off, the skin was exposed and disinfected, a longitudinal incision (about 1 cm) was cut along the longitudinal axis with a scalpel, and the dorsal neck muscle was bluntly dissected with scissors. To avoid bleeding, the deepest muscle attached to the bone was scraped with the back of the scalpel to expose the atlanto-occipital membrane. The cerebrospinal fluid was extracted directly through the occipital foramen. After extraction, the outer muscle and the skin were sutured. Sulfonamide powder was applied to the incision to prevent infection. After the cerebrospinal fluid was collected, an equivalent amount of sterilized saline was injected to maintain the pressure in the cerebrospinal cavity.

### 2. Reagent Preparation

(1) Positive control preparation: The MCP1 positive control was dissolved using 1 mL of deionized water and mixed thoroughly for later use.

(2) Scrubbing solution preparation: The scrubbing solution was diluted using deionized water at 1:25 to the working concentration.

(3) Luminescent reagent preparation: 15 minutes before the detection on the machine, the luminescent reagent A and the reagent B in the kit were mixed evenly at a volume ratio of 1:1 and stored away from light.

(4) MCP1 standard preparation: A 5000 μg/mL standard provided by the kit was diluted to a standard with the concentration of 500 μg/mL in a new EP tube using a calibrator diluent at 1:10 and then diluted to the standards with the concentrations of 250 μg/mL, 125 μg/mL, 62.5 pg/mL, 31.3 pg/mL, 15.6 pg/mL, and 7.81 pg/mL, respectively.

### 3. ELISA Detection

(1) 50 μL of detection diluent RD1W was added to the detection wells.

(2) The standards, the control sample, and the sample to be detected were sequentially added into the detection wells, sealed with the sealing film provided by the kit, and incubated for 2 hours at room temperature.

(3) After incubation, the sealing film was removed, the liquid was discarded, 400 μL of scrubbing solution was added to each well to wash the detection plate, the detection plate was washed four times, and the scrubbing solution was discarded.

(4) 100 μL of mouse MCP1 coupler was added to the detection wells, the detection wells were sealed with the sealing film, and incubation was performed at room temperature for 2 hours.

(5) Step (3) was repeated once.

(6) 100 μL of prepared luminescent reagent was added to the detection wells, and incubation was performed for 39 minutes at room temperature away from light.

(7) 100 μL of stop solution was added, and the detection plate was flicked gently to ensure that the mixture was mixed thoroughly.

(8) Plate reading: The optical density detection of all the wells was completed within 30 minutes.

(9) Calculation: The concentration was calculated quantitatively according to the formula provided by the kit.

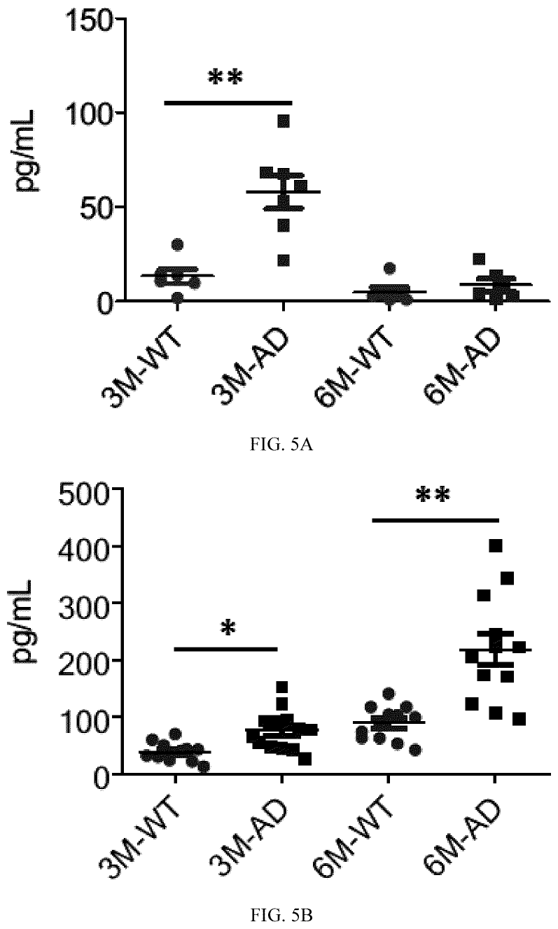

The detection results are shown in FIGS. 5A and 5B.

As can be seen from in FIG. 5A, the expression amount of the chemokine MCP1 was significantly increased in the cerebrospinal fluid of 3-month-old AD mice; and as can be seen from in FIG. 5B, the expression level of MCP1 was consistently increased in the peripheral blood of 3-month-old and 6-month-old AD mice. Since chemokines mainly play a pro-inflammatory role and induce immune cells to enter infection sites in the process of immune response, the expression level of chemokines was increased, indicating that peripheral blood chemokines control the chemotaxis of immune cells towards the site of neuroinflammation while playing an immune surveillance role during the progression of AD. The above results indicate that there is a correlation between inflammatory factors in the peripheral blood and the occurrence and development of neuroinflammation in the brain.

In summary, the expression of the chemokine MCP1 is significantly increased in the brain of AD mice, which in turn causes neuroinflammation; MCP1-mediated neuroinflammation occurs before 3 months of age and is accompanied by changes in the immune system and the exacerbation of neuroinflammation during the development of AD, indicating that MCP1 can be used as a marker in early screening of AD; since the detection result of MCP1 in the peripheral blood is consistent with the detection result of MCP1 in the cerebrospinal fluid, the screening and prevention of AD can be performed by directly detecting the peripheral blood, which is easy to operate and has lower risk and a higher application value.

The applicant has stated that although the detailed method of the present application is described through the examples described above, the present application is not limited to the detailed method described above, which means that the implementation of the present application does not necessarily depend on the detailed method described above. It is to be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

What is claimed is:

1. A marker of Alzheimer's disease, comprising a chemokine MCP1.

2. A method for detecting Alzheimer's disease for a purpose of non-disease diagnosis and/or treatment, comprising: detecting MCP1 of a sample.

3. The method for detecting Alzheimer's disease for the purpose of non-disease diagnosis and/or treatment according to claim 2, wherein the sample comprises cerebrospinal fluid and/or serum.

4. The method for detecting Alzheimer's disease for the purpose of non-disease diagnosis and/or treatment according to claim 2, wherein the detecting MCP1 of a sample comprises: detecting an expression level of MCP1 and/or determining a location of MCP1.

5. The method for detecting Alzheimer's disease for the purpose of non-disease diagnosis and/or treatment according to claim 4, wherein a method for detecting the expression level of MCP1 comprises real-time fluorescence quantitative PCR detection and/or ELISA detection.

6. The method for detecting Alzheimer's disease for the purpose of non-disease diagnosis and/or treatment according to claim 4, wherein a method for determining the location of MCP1 comprises immunofluorescence staining.

7. The method for detecting Alzheimer's disease for the purpose of non-disease diagnosis and/or treatment according to claim 2, further comprising a step of detecting any one or a combination of at least two of an activity of regulatory T cells, an activity of T helper cells, an aggregation level of amyloid beta (AB) plaques, an expression level of inflammatory factors or an expression level of cytokines of the sample.

8. A kit for detecting Alzheimer's disease, which detects the marker of Alzheimer's disease according to claim 1;
wherein the kit comprises any one of an immunofluorescence staining kit, a real-time fluorescence quantitative PCR kit or an ELISA kit.

9. The kit for detecting Alzheimer's disease according to claim 8, wherein the immunofluorescence staining kit comprises any one or a combination of at least two of a fixing agent, a dehydrating agent, an embedding agent, a cleaning liquid, a sealing agent, a primary antibody, a second antibody or a mounting medium.

10. The kit for detecting Alzheimer's disease according to claim 8, wherein the real-time fluorescence quantitative PCR kit comprises any one or a combination of at least two of an RNA extraction reagent, a reverse transcriptase, an amplification enzyme, a buffer, a primer, an RNA enzyme inhibitor, dNTPs or a fluorescent dye.

11. The kit for detecting Alzheimer's disease according to claim 8, wherein the ELISA kit comprises any one or a combination of at least two of an MCP1 conjugate, a luminescent reagent, a positive control, a standard substance, a diluent, a scrubbing solution or a stop solution.

12. (canceled)

13. A method for screening a drug for treating Alzheimer's disease, comprising using the marker of Alzheimer's disease according to claim 1.

14. The kit for detecting Alzheimer's disease according to claim 8, wherein the immunofluorescence staining kit comprises a fixing agent, a dehydrating agent, an embedding agent, a cleaning liquid, a sealing agent, a primary antibody, a second antibody, and a mounting medium.

15. The kit for detecting Alzheimer's disease according to claim 8, wherein the real-time fluorescence quantitative PCR kit comprises an RNA extraction reagent, a reverse transcriptase, an amplification enzyme, a buffer, a primer, an RNA enzyme inhibitor, dNTPs, and a fluorescent dye.

16. The kit for detecting Alzheimer's disease according to claim 8, wherein the ELISA kit comprises an MCP1 conjugate, a luminescent reagent, a positive control, a standard substance, a diluent, a scrubbing solution, and a stop solution.


SEQUENCE LISTING (2 sequences)

SEQ ID NO: 1 - DNA, 20 bp, Artificial sequence
  Forward primer
aggtgtcccaaagaagctgt

SEQ ID NO: 2 - DNA, 20 bp, Artificial sequence
  Reverse primer
acagaagtgcttgaggtggt